(19) United States
(12) Patent Application Publication
(10) Pub. No.: US 2022/0411839 A1
(43) Pub. Date: Dec. 29, 2022

(54) METHOD FOR PREPARING SORBITOL LIQUID AND LIQUID POLYOL BY USING MALTITOL RAFFINATE

(71) Applicant: Zhejiang Huakang Pharmaceutical Co., Ltd., Quzhou, Zhejiang (CN)

(72) Inventors: Baoxing MAO, Quzhou, Zhejiang (CN); Xiuxiu WANG, Quzhou, Zhejiang (CN); Deshui CHEN, Quzhou, Zhejiang (CN); Jiaxing LUO, Quzhou, Zhejiang (CN); Xinping CHENG, Quzhou, Zhejiang (CN)

(21) Appl. No.: 17/774,056

(22) PCT Filed: Feb. 7, 2021
(86) PCT No.: PCT/CN2021/075694

§ 371 Date: May 3, 2022

(30) Foreign Application Priority Data
Feb. 11, 2020 (CN) 202010087372.8

(51) Int. Cl. C12P 19/14; C12P 7/18

(52) U.S. Cl. CPC C12P 19/14; C12P 7/18; C12Y 302/01003

(57) ABSTRACT

The present invention provides a method of preparing a sorbitol liquid and a liquid polyol using a maltitol raffinate. The method includes the steps of: obtaining a saccharification liquid by adding glucoamylase to the maltitol raffinate for saccharification; obtaining a fermentation liquid by adding a dry yeast to the above saccharification liquid for fermentation; obtaining the sorbitol liquid and the liquid polyol by performing decolorization filtration and membrane separation for the above fermentation liquid. The present invention can fully utilize the maltitol raffinate to increase its added value. Further, the method is simple, practical and low in costs.

### TECHNICAL FIELD

The present invention relates to the field of recycling technologies of maltitol raffinates, and in particular to a method of preparing a sorbitol liquid and a liquid polyol using a maltitol raffinate.

### BACKGROUND

Maltitol is a disaccharide alcohol prepared by performing hydrogenation reduction for maltose. With advent and development of chromatography technology, a method of preparing a crystal maltitol by use of chromatographic separation has drawn broad attentions of people.

A given amount of chromatographic raffinate may be generated as a byproduct when a high purity maltitol component is prepared by use of chromatographic separation. The chromatographic raffinate byproduct has no high added value at present. The main components of the chromatographic raffinate are oligosaccharide alcohol (20-30%), sorbitol (20-30%), and maltitol (30-40%). The main process of maltitol is as follows:

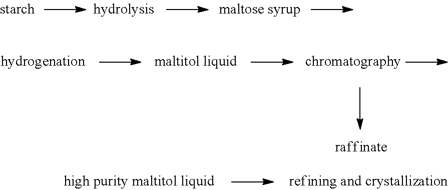

In order to increase the added value of the raffinate, a patent with a publication number CN104177229B provides a process of preparing a solid oligosaccharide alcohol and a solid sorbitol, in which the solid oligosaccharide alcohol and the solid sorbitol are prepared using a residue liquid of maltitol chromatographic separation such that an added-value use of the residue liquid of maltitol chromatographic separation can be realized. However, multiple components are obtained by performing separation and purification for the residue liquid based on chromatographic separation technology in this method, leading to increased complexity and increased difficulty of operation of a system. Further, one set of chromatography equipment is required, increasing the investment.

### SUMMARY

In order to solve the above technical problems, the present invention provides a method of preparing a sorbitol liquid and a liquid polyol using a maltitol raffinate, so as to effectively utilize the maltitol raffinate, and increase its added value. This method is simple, practical and low in costs.

The present invention is achieved by providing a method of preparing a sorbitol liquid and a liquid polyol using a maltitol raffinate. The method includes the following steps:

at step 1, obtaining a saccharification liquid by adding glucoamylase to the maltitol raffinate for saccharification;

at step 2, obtaining a fermentation liquid by adding a dry yeast to the above saccharification liquid for fermentation; and

at step 3, obtaining the sorbitol liquid and the liquid polyol by performing decolorization filtration and membrane separation for the above fermentation liquid.

Furthermore, the method includes the following steps:

at step 1, adding glucoamylase to the maltitol raffinate and performing saccharification at a temperature of 55-65° C. for 22-25 hours to obtain a saccharification liquid;

at step 2, adding a dry yeast to the above saccharification liquid and performing fermentation at a temperature of 30-40° C. for 60-65 hours to obtain a fermentation liquid; and

at step 3, obtaining a diluted liquid and a concentrated liquid by performing decolorizaiton filtration and membrane separation for the above fermentation liquid, wherein the diluted liquid is the sorbitol liquid, and the concentrated liquid is the liquid polyol.

Compared with prior art, the method of preparing a sorbitol liquid and a liquid polyol using a maltitol raffinate according to the present invention has the following advantages.

1) The components of the maltitol raffinate are fully utilized, increasing the added value of the product.

2) Before membrane separation, a given amount of glucoamylase is added for saccharification and then a given amount of yeast is added for fermentation, such that the contents of the total sugar and the reducing sugar in subsequent product are reduced, thus increasing the purities of the sorbitol liquid and the liquid polyol.

### DETAILED DESCRIPTION OF THE EMBODIMENTS

In order to make the technical problems, the technical solutions and the beneficial effects of the present invention clearer and more understandable, the present invention will be further described in combination with specific embodiments. It should be understood that the specific embodiments described herein are used only to explain the present invention rather than limit the present invention.

A preferred embodiment of the method of preparing a sorbitol liquid and a liquid polyol using a maltitol raffinate according to the present invention includes the following steps.

At step 1, glucoamylase is added to the maltitol raffinate for saccharification to obtain a saccharification liquid.

At step 2, a fermentation liquid is obtained by adding a dry yeast to the above saccharification liquid for fermentation.

At step 3, the sorbitol liquid and the liquid polyol are obtained by performing decolorization filtration and membrane separation for the above fermentation liquid.

Specifically, the preferred embodiment of the method of preparing a sorbitol liquid and a liquid polyol using a maltitol raffinate according to the present invention includes the following steps.

At step 1, glucoamylase with a mass being equal to 0.04-0.16% of a mass of the maltitol raffinate is added to the maltitol raffinate and saccharification is performed at a temperature of 55-65 ° C. for 22-25 hours to obtain a saccharification liquid, where oligosaccharide alcohol therein is hydrolyzed and a content of oligosaccharide alcohol measured in the saccharification liquid is reduced by 40-45%.

At step 2, a dry yeast with a mass being equal to 0.4-0.6% of the mass of the maltitol raffinate is added to the above saccharification liquid, and fermentation is performed at a temperature of 30-40° C. for 60-65 hours to obtain a fermentation liquid, wherein a content of a reducing sugar measured in the fermentation liquid is reduced to below 0.2%.

At step 3, a diluted liquid and a concentrated liquid are obtained by performing decolorizaiton filtration and membrane separation for the above fermentation liquid, wherein the diluted liquid is the sorbitol liquid, and the concentrated liquid is the liquid polyol.

The components of the diluted liquid and the concentrated liquid are measured as follows:

| | | | Oligosaccharide | Reducing |
|---|---|---|---|---|
| | Maltitol | Sorbitol | alcohol | surgar |
| Diluted | 9-14% | 70-80% | 5-10% | 0.15-0.20% |
| liquid | | | | |
| Concentrated | 40-50% | 13-18% | 35-40% | 0.07-0.15% |
| liquid | | | | |

The diluted liquid conforms to the state standard regarding sorbitol liquid in terms of components and can be directly used as a sorbitol liquid in a product. The concentrated liquid may be used as a multi-component liquid polyol in food processing due to a low content of reducing sugar and high contents of maltitol and oligosaccharide alcohol, so as to achieve the effects of moistening, increasing service life and increasing sweetness and the like. Because it is a sugar alcohol, it can also achieve the effect of reducing heat.

The method of preparing a sorbitol liquid and a liquid polyol using a maltitol raffinate according to the present invention will be further described in combination with the specific embodiments.

### Embodiment 1

The first embodiment of the method of preparing a sorbitol liquid and a liquid polyol using a maltitol raffinate according to the present invention includes the following steps.

At step 11, 1 kg of the maltitol raffinate was taken, 0.4g of glucoamylase was added, and saccharification was performed at a constant temperature of 60° C. for 24 hours to obtain a saccharification liquid, wherein a content of oligosaccharide alcohol measured in the saccharification liquid was reduced by 40%;

At step 12, 4g of a dry yeast was added to the above saccharification liquid, and fermentation was performed at a temperature of 30° C. for 65 hours to obtain a fermentation liquid; wherein a content of a reducing sugar was measured to be 0.15%.

At step 13, decolorization filtration was performed to remove the yeast in the fermentation liquid and then membrane separation was performed to obtain a diluted liquid and a concentrated liquid respectively, wherein the diluted liquid was the sorbitol liquid and the concentrated liquid was the liquid polyol.

The components of the diluted liquid and the concentrated liquid were measured as follows:

| | | | Oligosaccharide | Reducing |
|---|---|---|---|---|
| | Maltitol | Sorbitol | alcohol | surgar |
| Diluted | 10% | 75% | 10% | 0.17% |
| liquid | | | | |
| Concentrated | 45% | 13% | 35% | 0.09% |
| liquid | | | | |

### Embodiment 2

The second embodiment of the method of preparing a sorbitol liquid and a liquid polyol using a maltitol raffinate according to the present invention includes the following steps.

At step 21, 1 kg of the maltitol raffinate was taken, lg of glucoamylase was added, and saccharification was performed at a constant temperature of 55° C. for 25 hours to obtain a saccharification liquid, wherein a content of oligosaccharide alcohol measured in the saccharification liquid was reduced by 45%.

At step 22, 5 g of a dry yeast was added to the above saccharification liquid and fermentation was performed at a temperature of 40° C. for 60 hours to obtain a fermentation liquid, wherein a content of a reducing sugar was measured to be 0.12%.

At step 23, decolorization filtration was performed to remove the yeast in the fermentation liquid and then membrane separation was performed to obtain a diluted liquid and a concentrated liquid respectively, wherein the diluted liquid was the sorbitol liquid and the concentrated liquid was the liquid polyol.

The components of the diluted liquid and the concentrated liquid were measured as follows:

| | | | Oligosaccharide | Reducing |
|---|---|---|---|---|
| | Maltitol | Sorbitol | alcohol | surgar |
| Diluted | 11% | 78% | 8% | 0.15% |
| liquid | | | | |
| Concentrated | 43% | 15% | 37% | 0.1% |
| liquid | | | | |

### Embodiment 3

The third embodiment of the method of preparing a sorbitol liquid and a liquid polyol using a maltitol raffinate according to the present invention includes the following steps.

At step 31, 1 kg of the maltitol raffinate was taken, 1.6g of glucoamylase was added, and saccharification was performed at a constant temperature of 65° C. for 22 hours to obtain a saccharification liquid, wherein a content of oligosaccharide alcohol measured in the saccharification liquid was reduced by 43%.

At step 32, 6 g of a dry yeast was added to the above saccharification liquid and fermentation was performed at a temperature of 35° C. for 63 hours to obtain a fermentation liquid, wherein a content of a reducing sugar was measured to be 0.14%.

At step 33, decolorization filtration was performed to remove the yeast in the fermentation liquid and then membrane separation was performed to obtain a diluted liquid and a concentrated liquid respectively, wherein the diluted liquid was the sorbitol liquid and the concentrated liquid was the liquid polyol.

The components of the diluted liquid and the concentrated liquid were measured as follows:

| | | | Oligosaccharide | Reducing |
|---|---|---|---|---|
| | Maltitol | Sorbitol | alcohol | surgar |
| Diluted | 13% | 77% | 8% | 0.16% |
| liquid | | | | |
| Concentrated | 48% | 15% | 36% | 0.09% |
| liquid | | | | |

The above descriptions are merely made to the preferred embodiments of the present invention and shall not be intended to limit the present invention. Any modifications, equivalent substitutions and improvements and the like made within the spirit and principle of the present invention shall all fall within the scope of protection of the present invention.

1. A method of preparing a sorbitol liquid and a liquid polyol using a maltitol raffinate, comprising:
at step 1, obtaining a saccharification liquid by adding glucoamylase to the maltitol raffinate for saccharification;
at step 2, obtaining a fermentation liquid by adding a dry yeast to the saccharification liquid for fermentation; and
at step 3, obtaining the sorbitol liquid and the liquid polyol by performing decolorization filtration and membrane separation for the fermentation liquid.

2. The method of claim 1, further comprising:
at step 1, performing saccharification at a temperature of 55-65° C. for 22-25 hours to obtain the saccharification liquid;
at step 2, performing fermentation at a temperature of 30-40° C. for 60-65 hours to obtain the fermentation liquid;
at step 3, obtaining a diluted liquid and a concentrated liquid by performing decolorizaiton filtration and membrane separation for the fermentation liquid, wherein the diluted liquid is the sorbitol liquid, and the concentrated liquid is the liquid polyol.

3. The method of claim 2, wherein at step 1, the glucoamylase is added in an amount of 0.04-0.16% of the mass of the maltitol raffinate.

4. The method of claim 2, wherein at step 1, a content of oligosaccharide alcohol measured in the saccharification liquid is reduced by 40-45%.

5. The method of claim 2, wherein at step 2. the dry yeast is added in an amount of 0.4-0.6% of the mass of the maltitol raffinate.

6. The method of claim 2, wherein at step 2, the amount of a reducing sugar measured in the fermentation liquid is reduced to below 0.2%.

7. The method of claim 2, wherein
step 1 comprises adding 0.4 g of glucoamylase into 1 kg of the maltitol raffinate, and performing saccharification at a temperature of 60° C. for 24 hours to obtain the saccharification liquid, wherein a content of oligosaccharide alcohol measured in the saccharification liquid is reduced by 40%;
step 2 comprises adding 4 g of the dry yeast to the saccharification liquid, and performing fermentation at a temperature of 30° C. for 65 hours to obtain the fermentation liquid; and
step 3 comprises performing decolorization filtration to remove the dry yeast from the fermentation liquid and then performing membrane separation to obtain a diluted liquid and a concentrated liquid respectively, wherein the diluted liquid is the sorbitol liquid and the concentrated liquid is the liquid polyol.

8. The method of claim 2, wherein
step 1 comprises adding 1 g of glucoamylase into 1 kg of the maltitol raffinate, and performing saccharification at a temperature of 55° C. for 25 hours to obtain the saccharification liquid, wherein a content of oligosaccharide alcohol measured in the saccharification liquid is reduced by 45%;
step 2 comprises adding 5 g of the dry yeast to the saccharification liquid and performing fermentation at a temperature of 40° C. for 60 hours to obtain the fermentation liquid; and
step 3 comprises performing decolorization filtration to remove the dry yeast from the fermentation liquid and then performing membrane separation to obtain a diluted liquid and a concentrated liquid respectively, wherein the diluted liquid is the sorbitol liquid and the concentrated liquid is the liquid polyol.

9. The method of claim 2, wherein
step 1 comprises adding 1.6 g of glucoamylase into 1 kg of the maltitol raffinate, and performing saccharification at a temperature of 65° C. for 22 hours to obtain the saccharification liquid, wherein a content of oligosaccharide alcohol measured in the saccharification liquid is reduced by 43%;
step 2 comprises adding 6 g of the dry yeast to the saccharification liquid and performing fermentation at a temperature of 35° C. for 63 hours to obtain the fermentation liquid;
step 3 comprises performing decolorization filtration to remove the dry yeast from the fermentation liquid and then performing membrane separation to obtain a diluted liquid and a concentrated liquid respectively, wherein the diluted liquid is the sorbitol liquid and the concentrated liquid is the liquid polyol.
